(19) United States
(12) Patent Application Publication
(10) Pub. No.: US 2019/0262323 A1
(43) Pub. Date: Aug. 29, 2019

(54) COMPOSITIONS AND METHODS FOR TREATING NEURODEGENERATIVE DISORDERS

(71) Applicant: GEORGETOWN UNIVERSITY, Washington, DC (US)

(72) Inventor: Charbel Moussa, Germantown, MD (US)

(73) Assignee: GEORGETOWN UNIVERSITY, Washington, DC (US)

(21) Appl. No.: 16/345,995

(22) PCT Filed: Oct. 31, 2017
(86) PCT No.: PCT/US2017/059206

§ 371 Date: Apr. 29, 2019

Related U.S. Application Data
(60) Provisional application No. 62/414,916, filed on Oct. 31, 2016.

(51) Int. Cl. A61K 31/4365; A61P 25/28; C07D 495/04; A61K 9/00; A61K 31/519; A61K 31/5377

(52) U.S. Cl. CPC A61K 31/4365; A61P 25/28; C07D 495/04; A61K 45/06; A61K 9/0019; A61K 31/519; A61K 31/5377; A61K 9/0053

(57) ABSTRACT

Provided herein are methods of treating or preventing a neurodegenerative disease in a subject using an inhibitor of discoidin domain receptor (DDR) tyrosine kinase.

This application claims priority to U.S. Provisional Application No. 62/414,916, filed Oct. 31, 2016, which is hereby incorporated in its entirety by this reference.

### BACKGROUND

Neurodegenerative diseases include genetic and sporadic disorders associated with progressive nervous system dysfunction. These diseases are characterized by progressive deterioration of nerve cells or nerve cell function. It has been estimated that one of four Americans will develop a neurodegenerative condition in their lifetimes. Generally, however, the underlying mechanisms causing the conditions are not well understood and few effective treatment options are available for preventing or treating neurodegenerative diseases.

### SUMMARY

Provided herein are methods of treating or preventing a central nervous system neurodegenerative disease in a subject. The methods comprise administering to the subject with the neurodegenerative disease or at risk of developing the neurodegenerative disease an effective amount of a compound having the formula of Formula I

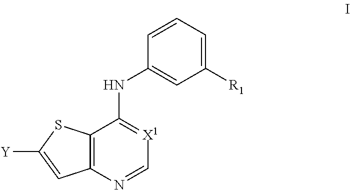

wherein,
X₁ is N or CH;
R₁ is —OH or —OCH₃;
Y is C₆₋₁₀ aryl substituted with R², or C₅₋₁₀ heteroaryl substituted with R² or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, di C₁₋₃ alkylamino, di C₁₋₃ alkylamino, hydroxyl C₁₋₃ alkylamino, carboxy C₁₋₃ alkylamino, C₃₋₆ cycloalkyl C₁₋₃ alkylamino, pyrrolidinyl, hydroxyl pyrrolidinyl, hydroxyl C₁₋₃ alkylpyrolidinyl, carboxypyrrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
   or an isomer or pharmaceutically acceptable salt thereof.

Also provided are methods of inhibiting or preventing toxic protein aggregation in a neuron. The methods comprise contacting the neuron with an effective amount of a composition comprising a compound having the formula of Formula I

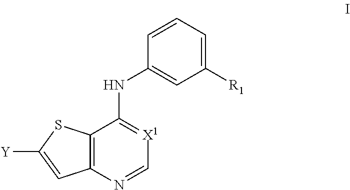

wherein,
X₁ is N or CH;
R₁ is —OH or —OCH₃;
Y is C₆₋₁₀ aryl substituted with R₂, or C₅₋₁₀ heteroaryl substituted with R₂ or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
   or an isomer or pharmaceutically acceptable salt thereof.

Further provided are methods of rescuing a neuron from neurodegeneration. The methods comprise contacting the neuron with an effective amount of a composition comprising a compound having the formula of Formula I

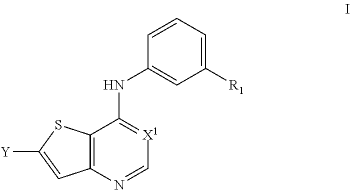

wherein,
X₁ is N or CH;
R₁ is —OH or —OCH₃;
Y is C₆₋₁₀ aryl substituted with R₂, or C₅₋₁₀ heteroaryl substituted with R₂ or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
   or an isomer or pharmaceutically acceptable salt thereof.

### DESCRIPTION OF FIGURES

FIG. 1 shows the results of a human Tau [pS396] solid phase sandwich ELISA performed on brain tissue homogenates from transgenic APP mice. Mice treated with LCB 03-0110 at 10 mg/kg, 5 mg/kg, 2.5 mg/kg, and 1.25 mg/kg had reduced levels of phosphorylated Tau. ** Indicates significant difference as compared to APP mice treated with DMSO with P<0.01. Bars are mean±SEM, one-way ANOVA.

FIG. 2A shows the results of a human Tau [pS396] solid phase sandwich ELISA performed on brain tissue homogenates from transgenic APP mice. Mice treated with LCB 03-0110 at 10 mg/kg, 5 mg/kg, 2.5 mg/kg, and 1.25 mg/kg had reduced ratios of Tau (phosphorylated Tau (pS396)/total Tau)(pg/ml). ** Indicates significant difference than APP mice treated with DMSO with P<0.01. Bars are mean±SEM, one-way ANOVA.

FIG. 2B shows the results of a human Tau [pS396] solid phase sandwich ELISA performed on brain tissue homogenates from transgenic APP mice. Mice treated with LCB 03-0110 at 10 mg/kg, 5 mg/kg, 2.5 mg/kg, and 1.25 mg/kg had reduced ratios of Tau (phosphorylated Tau (pS396)/total Tau)(%). ** Indicates significant difference than APP mice treated with DMSO with P<0.01. Bars are mean±SEM, one-way ANOVA.

FIG. 2C shows the results of a human Tau [pS396] solid phase sandwich ELISA performed on brain tissue homogenates from transgenic APP mice. Mice treated with LCB 03-0110 at 10 mg/kg, 5 mg/kg, 2.5 mg/kg, and 1.25 mg/kg did not exhibit reduced total Tau levels.

FIG. 3A shows the results of probing soluble fractions from brain tissue homogenates of transgenic APP mice treated with LCB 03-0110 at 2.5 mg/kg, and 1.25 mg/kg. Soluble fractions were probed with mouse monoclonal anti-6E10 (1:1000), rabbit polyclonal anti-Ab42 (1:1000), mouse monoclonal anti-AT180 (1:1000), rabbit polyclonal anti-Beclin-1 (1:1000), rabbit polyclonal anti-Atg7 (1:1000), rabbit polyclonal anti-Atg12 (1:1000), rabbit polyclonal anti-LC3B (1:1000) and rabbit polyclonal anti-actin (1:1000). LCB 03-0110 reduces amyloid-beta fragments and hyperphosphoryalted tau. Levels of APP/Aβ, Aβ₄₂, p-tau (AT180) were reduced following daily treatment with DMSO, 1.25, or 2.5 mg/kg LCB-03-0110 after one week, as measured by Western blot in APP mice (actin used as a loading control) (left panel). Densitomety analysis (n=5) showed that LCB-03-110 significantly reduced the levels of amyloid in 3 mutant APP mice that express both Ab42 and hyperphosphorylated tau as early as 4 months of age (right panel). These data indicate that LCB-03-110 can reduce the level of amyloid proteins at a concentration of about 2.5 mg/kg or lower. One way ANOVAAPP mice, mean±standard error of the mean. Asterisks denote a significant difference (*** p>0.001).

FIG. 3B is a Western blot of Beclin, Atg7, Atg12, LC3-I, and LC3-II levels in APP mice treated with DMSO, 1.25, or 2.5 mg/kg LCB-03-0110. LCB-03-0110 stimulates autophagy and clears autophagosomes.

FIG. 3C is a densitometry analysis of LC3-II levels (n=5) showing that LCB-03-110 significantly reduces the level of LCB-II/LCB-I suggesting that it activates autophagic clearance. These data indicate that low doses, for example, doses of 2.5 mg/kg or less, of LCB-03-0110 can induce autophagy and clear beta-amyloid and tau as indicated in FIG. 3A. Asterisks denote a significant difference (*** p>0.001).

FIG. 3D shows a MILLIPLEX® analysis of phosphorylated mTOR levels. LCB-03-0110 stimulates autophagy and clears autophagosomes without altering mTOR. Significance was assessed by one-way ANOVA. Data are shown as mean fluorescent intensity (MFI)±standard error of the mean. Asterisks denote a significant difference (*** p>0.001).

FIG. 4A shows that administration of 2.5 mg/kg or 1.25 mg/kg LCB-03-0110 does not alter RANTES levels. Data are shown as mean±standard error of the mean.

FIG. 4B shows that administration of 2.5 mg/kg or 1.25 mg/kg LCB-03-0110 reduces VEGF-A to control levels. Data are shown as mean±standard error of the mean.

FIGS. 5A-5G show that administration of LCB-03-0110 reduces brain inflammation, as evidenced by a reduction, in granulocyte colony-stimulating factor (G-CSF) (FIG. 5A), granulocyte-macrophage colony-stimulating factor (GM-CSF)(FIG. 5B), macrophage colony-stimulating factor (M-CSF) (FIG. 5C), macrophage inflammatory factor 1 alpha (MIP-1α) (FIG. 5D), macrophage inflammatory factor 1 beta (MIP-1β) (FIG. 5E), macrophage inflammatory protein 2 (MIP-2) (FIG. 5F), and macrophage inflammatory protein 1 (MIP-1) (FIG. 5G).

FIG. 6 shows that by day three of training in a Morris water maze, mice treated with 2.5 mg/kg LCB-03110 for three weeks had a 30% reduction in the average time to find a submerged platform. The data are shown as an average of three trials.

FIG. 7A shows that solube human Aβ₄₀ and Aβ₄₂ levels were significantly reduced following daily treatment with 2.5 mg/kg LCB-03-0110 for 3 weeks. Significance was assessed by an unpaired, two-tailed T-test. Data are shown as mean±standard error of the mean. Asterisks denote significance (* p>0.05, **** p>0.0001) FIG. 7B shows that insoluble human Aβ₄₂ levels were significantly reduced in the brains of APP mice following daily treatment with 2.5 mg/kg LCB-03-0110 for 3 weeks. Significance was assessed by an unpaired, two-tailed T-test. Data are shown as mean±standard error of the mean. Asterisks denote significance (* p>0.05, **** p>0.0001).

FIG. 8A shows that LCB-03-0110 penetrates the blood-brain barrier. Brain response values for LCB-03-0110 in APP mice (Area/IS Area), after administration of 10 mg/kg, 5 mg/kg, 2.5 mg/kg or 1.25 mg/kg LCB-03-0110, are shown. In these studies, 10 mg/kg LCB-03-0110 yielded the highest drug concentration in the brain and LCB-03-110 peaked in the brain at about 1 hour and was washed out completely at about 4 hours.

FIG. 8B shows serum response values for LCB-03-0110 in APP mice (Area/IS Area) after administration of 10 mg/kg, 5 mg/kg, 2.5 mg/kg or 1.25 mg/kg LCB-03-0110.

FIG. 8C shows the ratio of brain to serum response values for LCB-03-0110 in APP mice (Area/IS Area). In these studies, 2.5 mg/kg LCB-03-110 yielded the highest plasma:brain ratio, with a Tmax at about 2 hours (FIG. 8C).

### DETAILED DESCRIPTION

Provided herein are methods of treating or preventing a neurodegenerative disease. The neurodegenerative disease can be a neurodegenerative disease of the central nervous system. These include, but are not limited to, amyotrophic lateral sclerosis, Alzheimer's disease, frontotemporal dementia, frontotemporal dementia with TDP-43, frontotemporal dementia linked to chromosome-17, Pick's disease, Huntington's disease, mild cognitive impairment, an α-synucleinopathy (e.g., Parkinson's disease, Lewy body disease, multiple system atrophy), a Tauopathy, progressive supranuclear palsy, and cortico-basal degeneration. The neurodegenerative disease can also be a secondary neurodegenerative disease induced by a traumatic brain injury, stroke or an infection, for example, a bacterial or a viral infection (e.g., HIV, Herpes simplex virus (HSV)). The methods comprise administering to the subject with the neurodegenerative disease or at risk of developing the neurodegenerative disease an effective amount of a compound having the formula of Formula I

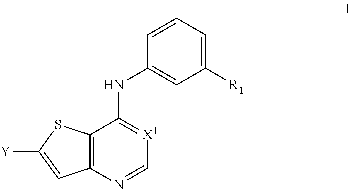

wherein,
X₁ is N or CH;
R¹ is —OH or —OCH₃;
Y is C₆₋₁₀ aryl substituted with R², or C₅₋₁₀ heteroaryl substituted with R² or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, di C₁₋₃ alkylamino, di C₁₋₃ alkylamino, hydroxyl C₁₋₃ alkylamino, carboxy C₁₋₃ alkylamino, C₃₋₆ cycloalkyl C₁₋₃ alkylamino, pyrrolidinyl, hydroxyl pyrrolidinyl, hydroxyl C₁₋₃ alkylpyrolidinyl, carboxypyrrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
   or an isomer or pharmaceutically acceptable salt thereof.

In the methods of the present invention, R³ of a compound having Formula I can be —H, —CN, halogen, C₁₋₃ alkyl, C₁₋₃ alkoxy, phenyl, pyridinyl, amino, ethylamino, diethylamino, pyrrolidinyl, piperidinyl, piperazinyl, morpholinyl or any one selected from the group consisting of the structural formula shown below

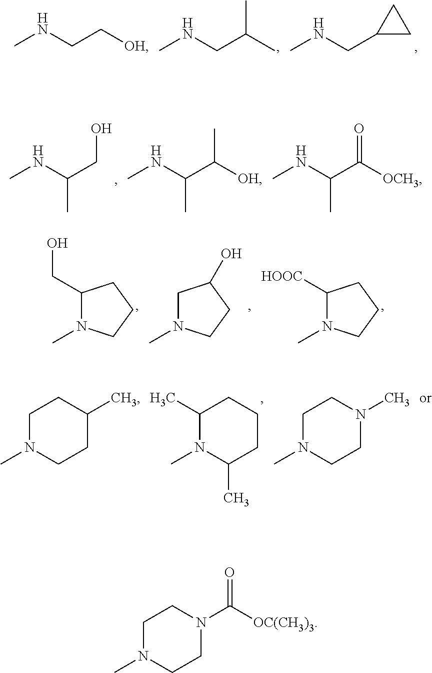

As used herein, the term pharmaceutically acceptable salt refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, trifluoroacetic acid, undecanoate, valerate salts, and the like.

In the methods provided herein, the compound having the formula of Formula I can be a compound having the formula of Formula II as shown below.

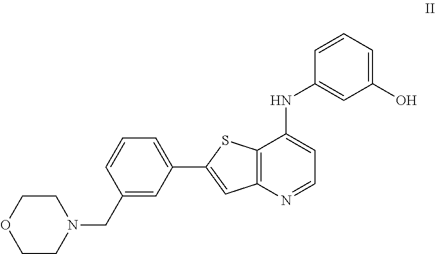 [SMILES: Oc1cccc(Nc2ccnc3cc(-c4cccc(CN5CCOCC5)c4)sc23)c1]

The compound represented by Formula II is also known as LCB 03-0110 or 3-(2-(3-(morpholinomethyl)phenyl)thieno[3,2-b]pyridine-7-ylamino)phenol. Methods of making a compound having the formula of Formula I or the formula of Formula II are provided in U.S. Patent Application Publication No. 2013/0072482, which is hereby incorporated herein in its entirety by this reference. The methods include the use of compounds having Formula I or derivatives thereof that cross the blood brain barrier. Any of the compounds described herein can be modified to enhance blood-brain barrier permeability. Optionally, one or more of the compounds described herein, including those having Formula I, can be administered with an agent that enhances the blood brain barrier permeability of the compound(s).

Compounds having the formula of Formula I or Formula II are inhibitors of the discoidin domain receptor (DDR) tyrosine kinases, which include DDR1 and DDR2. Both DDR1 and DDR2 serve as receptors for several collagen types. These receptors have been found to modulate cell proliferation and metalloprotease expression in response to collagen stimulation.

The methods provided herein optionally include selecting a subject with a neurodegenerative disease or at risk for developing a neurodegenerative disease. One of skill in the art knows how to diagnose a subject with or at risk of developing a neurodegenerative disease. For example, one or more of the follow tests can be used: a genetic test (e.g., identification of a mutation in TDP-43 gene) or familial analysis (e.g., family history), central nervous system imaging (e.g., magnetic resonance imaging and positron emission tomography), clinical or behavioral tests (e.g., assessments of muscle weakness, tremor, or memory), or laboratory tests.

The method optionally further includes administering a second therapeutic agent to the subject. The second therapeutic agent is selected from the group consisting of levadopa, a dopamine agonist, an anticholinergic agent, a cholinergic agent (e.g., 5-hydroxytryptamine (5-HT) inhibitors), a monoamine oxidase inhibitor, a COMT inhibitor, donepezil, memantine, risperidone, amantadine, rivastigmine, an NMDA antagonist, an acetylcholinesterase inhibitor, a cholinesterase inhibitor, riluzole, an anti-psychotic agent, an antidepressant, a second tyrosine kinase inhibitor (e.g., nilotinib, bosutinib or pazopanib), and tetrabenazine. In the methods where a second tyrosine kinase inhibitor is administered, the second tyrosine kinase inhibitor can be a tyrosine kinase inhibitor that does not inhibit DDR1 and/or DDR2 or has decreased selectivity for DDR1 and/or DDR2, as compared to a compound of Formula I.

Also provided herein is a method of inhibiting or preventing toxic protein aggregation in a neuron and/or rescuing a neuron from degeneration. The method includes contacting the neuron with an effective amount of a compound of Formula I. Optionally, the compound having Formula I is a compound having Formula II. The toxic protein aggregate optionally comprises one or more of an amyloidogenic protein, alpha-synuclein, tau, or TDP-43. By amyloidogenic protein is meant a peptide, polypeptide, or protein that has the ability to aggregate. An example of an amyloidogenic protein is β-amyloid.

The contacting is performed in vivo or in vitro. The in vivo method is useful in treating a subject with or at risk of developing toxic protein aggregates and comprises administering the compound of Formula I to the subject as described below. The in vitro method is useful, for example, in treating neural cells prior to transplantation. In such case, the compound of Formula I is generally added to a culture medium. Optionally, the target neurons are contacted with a second therapeutic agent as described above.

The term effective amount, as used throughout, is defined as any amount, for example, an amount of a compound of Formula I, necessary to produce a desired physiologic response. For example, the dosage is optionally less than about 10 mg/kg and can be less than about 9.5, 9, 8.5, 8, 7.5, 7, 6.5, 6, 5.5, 5, 4.5, 4, 3.5, 3, 2.5, 2, 1.5, 1.25, 1.0, 0.9, 0.8, 0.7, 0.6, 0.5, 0.4, 0.3, 0.2, 0.1 mg/kg or any dosage in between these amounts. The dosage can range from about 0.1 mg/kg to about 10 mg/kg, from about 0.1 mg/kg to about 9 mg/kg, from about 0.1 mg/kg to about 8 mg/kg, from about 0.1 mg/kg to about 7 mg/kg, from about 0.1 mg/kg to about 6 mg/kg, from about 0.1 mg/kg to about 5 mg/kg, from about 0.1 mg/kg to about 4 mg/kg, from about 0.1 mg/kg to about 3 mg/kg, from about 0.1 mg/kg to about 2.5 mg·kg, from about 0.1 mg/kg to about 2 mg/kg, from about 0.1 mg/kg to about 1.5 mg/kg, from about 0.1 mg/kg to about 1 mg/kg, or from about 0.1 mg/kg to about 0.5 mg/kg. One of skill in the art would adjust the dosage as described below based on specific characteristics of the inhibitor and the subject receiving it.

Also provided are compositions comprising the compound having Formula I, for example, the compound of Formula II. The composition can comprise a single unit dose of a compound of Formula I, for example, a single unit dose of about 10 mg/kg or less, of about 5 mg/kg or less, of about 2.5 mg/kg or less or about 1.5 mg/kg or less of a compound having Formula II (LCB-03-110) or a pharmaceutically acceptable salt thereof. Packages including one or multiple, single unit doses of a compound having Formula I, for example, multiple, single unit doses of a compound of Formula II are also provided. The package can further comprise single or multiple unit doses of one or more second therapeutic agents described herein.

Effective amounts and schedules for administering the compound of Formula I can be determined empirically and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the type of inhibitor, the species, age, body weight, general health, sex and diet of the subject, the mode and time of administration, rate of excretion, drug combination, and severity of the particular condition and can be determined by one of skill in the art. The dosage can be adjusted by the individual physician in the event of any contraindications. Dosages can vary, and can be administered in one or more dose administrations daily.

The DDR inhibitors described herein can be provided in a pharmaceutical composition. These include, for example, a pharmaceutical composition comprising a therapeutically effective amount of one or more DDR inhibitors and a pharmaceutical carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, N.J.), polyethylene glycol (PEG), and PLURONICS™ (BASF; Florham Park, N.J.).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

The compositions are administered in a number of ways depending on whether local or systemic treatment is desired, and on the area to be treated. The compositions are administered via any of several routes of administration, including orally, parenterally, intravenously, intraperitoneally, intracranially, intraspinally, intrathecally, intraventricularly, intramuscularly, subcutaneously, intracavity or transdermally. Pharmaceutical compositions can also be delivered locally to the area in need of treatment, for example by topical application or local injection. Effective doses for any of the administration methods described herein can be extrapolated from dose-response curves derived from in vitro or animal model test systems.

Throughout, treat, treating, and treatment refer to a method of reducing or delaying one or more effects or symptoms of a neurodegenerative disease or disorder. The subject can be diagnosed with a disease or disorder. Treatment can also refer to a method of reducing the underlying pathology rather than just the symptoms. The effect of the administration to the subject can have the effect of but is not limited to reducing one or more symptoms of the neurodegenerative disease or disorder, a reduction in the severity of the neurological disease or injury, the complete ablation of the neurological disease or injury, or a delay in the onset or worsening of one or more symptoms. For example, a disclosed method is considered to be a treatment if there is about a 10% reduction in one or more symptoms of the disease in a subject when compared to the subject prior to treatment or when compared to a control subject or control value. Thus, the reduction can be about a 10, 20, 30, 40, 50, 60, 70, 80, 90, 100%, or any amount of reduction in between.

As utilized herein, by prevent, preventing, or prevention is meant a method of precluding, delaying, averting, obviating, forestalling, stopping, or hindering the onset, incidence, severity, or recurrence of the neurodegenerative disease or disorder. For example, the disclosed method is considered to be a prevention if there is a reduction or delay in onset, incidence, severity, or recurrence of neurodegeneration or one or more symptoms of neurodegeneration (e.g., tremor, weakness, memory loss, rigidity, spasticity, atrophy) in a subject susceptible to neurodegeneration as compared to control subjects susceptible to neurodegeneration that did not receive a compound having the Formula I. The disclosed method is also considered to be a prevention if there is a reduction or delay in onset, incidence, severity, or recurrence of neurodegeneration or one or more symptoms of neurodegeneration in a subject susceptible to neurodegeneration after receiving a compound having the Formula I as compared to the subject's progression prior to receiving treatment. Thus, the reduction or delay in onset, incidence, severity, or recurrence of neurodegeneration can be about a 10, 20, 30, 40, 50, 60, 70, 80, 90, 100%, or any amount of reduction in between.

As used throughout, by subject is meant an individual. Preferably, the subject is a mammal such as a primate, and, more preferably, a human. Non-human primates are subjects as well. The term subject includes domesticated animals, such as cats, dogs, etc., livestock (for example, cattle, horses, pigs, sheep, goats, etc.) and laboratory animals (for example, ferret, chinchilla, mouse, rabbit, rat, gerbil, guinea pig, etc.). Thus, veterinary uses and medical formulations are contemplated herein.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### Examples

### DDR1 and DDR2 Knockdown Mice

shRNAs were designed to specifically knock down DDR1 and DDR2 receptors, and study their effects on clearance of misfolded protein accumulation in neurodegeneration. DDR1/2 knockdown in vitro and in vivo was found to reduce the levels of alpha-Synuclein, beta-amyloid and Tau, and to prevent cell death caused by accumulation of these misfolded proteins in culture. These shRNAs were then injected into animal models in vivo. In a A53T mouse model that expresses human alpha-Synuclein and murine Tau, DDR1/2 knockdown reduced the levels of Tau and alpha-Synuclein and also reduced the level of inflammation in these animals. These data indicated that DDR1/2 inhibition is a therapeutic target for neurodegenerative diseases. A known DDR inhibitor, LCB-03-0110, was tested in cell culture. Low doses (0.1-3 nM) of LCB-03-0110 reduced the levels of beta-amyloid, synuclein and Tau in cell culture.

Treatment of Amyloid Precursor Protein (APP) Transgenic Mice with LCB 03-0110

Animal Treatment:

Transgenic mice harboring the Swedish, Dutch, and Iowa mutation for human amyloid beta-precursor protein were treated with intraperitoneal (IP) injections of 10 mg/kg, 5 mg/kg, 2.5 mg/kg, 1.25 mg/kg of LCB 03-0110 or 30 μL of dimethylsulfoxide (DMSO) every day for 1 week. LCB 03-0110 is a potent inhibitor of discoidin domain receptor 2 (DDR2) family tyrosine kinases. All animal experiments were conducted in full compliance with the recommendations of Georgetown University Animal Care and Use Committee (GUAUC). Fifteen mice were used for brain and blood extractions, and fifteen mice were used for drug treatments. Four transgenic APP mice were treated with DMSO, four transgenic APP mice were treated with 10 mg/kg of LCB 03-0110, four transgenic APP mice were treated with 5 mg/kg of LCB 03-0110, four transgenic APP mice were treated with 2.5 mg/kg of LCB 03-0110, and three transgenic APP mice were treated with 1.25 mg/kg of LCB 03-0110. Seven transgenic APP mice were treated with DMSO, and 7 transgenic APP mice were treated with 2.5 mg/kg of LCB.

All graphs and statistical analyses were performed in Graph Pad Prism Software (Graph Pad Prism Software, Inc. CA. USA).

Tissue Collection and Homogenization:

Animals were deeply anesthetized with a mixture of Xylazine and Ketamine (1:8). Whole blood (500 μl) was collected via cardiac puncture and centrifuged at 2000×g to precipitate blood cells. The serum was collected for mass spectrometric analysis. To wash out the remaining blood from vessels and reduce contamination, animals were perfused with 25 ml of 1X phosphate buffered saline (PBS) for 5 min and the brains were collected. A hemisphere was frozen and stored at −80° C. for mass spectrometry studies and the other hemisphere was immediately homogenized in 2.0 ml lysis buffer (containing protease and phosphatase inhibitors) and labeled as a soluble protein fraction.

Protein Extraction:

After removing the soluble supernatant, the tissue pellet was washed with 1×STEN buffer. The pellet was resuspended in 750 ul of 70% formic acid and incubated for 30 min at room temperature followed by centrifugation at 28,000 g at 4° C. for 1 hour. The supernatant was collected as the insoluble fraction. Samples from the 70% formic acid fraction were stored at −80° C. and neutralized with 1M Tris-base (1:20) immediately before use.

Enzyme-Linked Immunosorbent Assays:

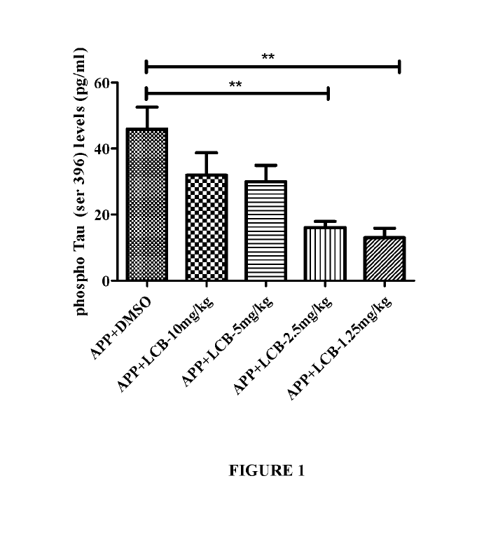

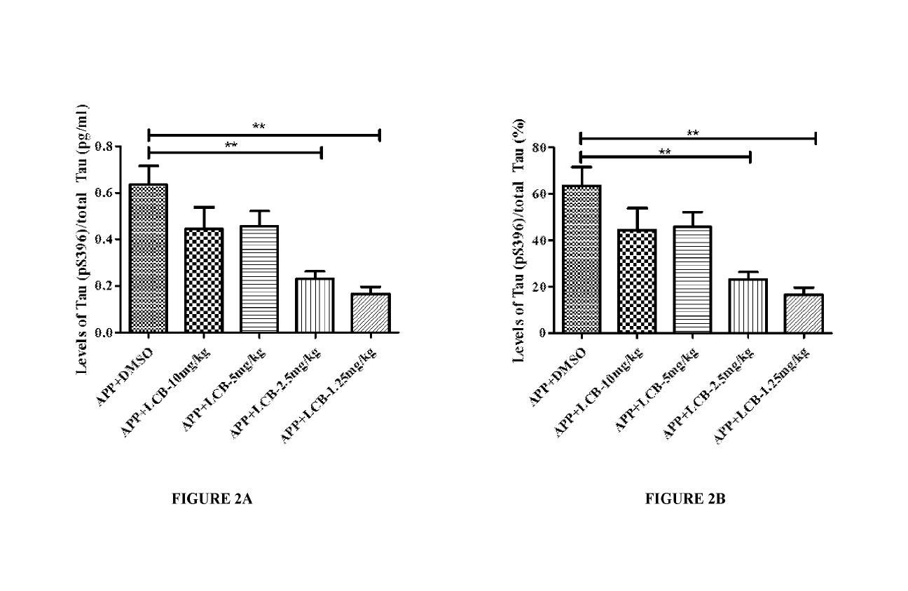

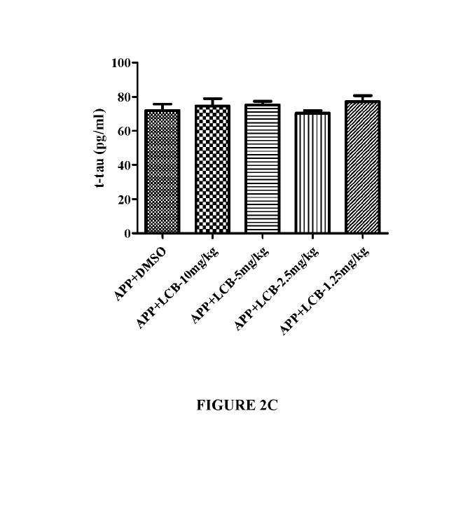

Human Tau [pS396] solid phase sandwich ELISA was performed on brain tissue homogenates from transgenic APP mice treated with LCB 03-0110 at 10 mg/kg, 5 mg/kg, 2.5 mg/kg, and 1.25 mg/kg. A monoclonal Tau capture antibody was coated onto micro-wells. 50 μl of soluble lysate was added to each well, allowing the Tau [pS396] antigen to bind to the immobilized capture antibody, and incubated for two hours at room temperature. After the two hour incubation, samples were washed and incubated with 100 μl of rabbit monoclonal tau [pS396] detection antibody and incubated for one hour at room temperature. Following extensive washing, a 100 μl of horseradish peroxidase labeled anti-rabbit IgG was added to each well and incubated for thirty minutes at room temperature. Samples were washed to remove all unbound enzyme. 100 μl of 3,3,5,5′ tetramethylbenzine (TMB), a horseradish peroxidase (HRP) substrate, was added to develop color. The magnitude of the absorbance for this developed color was proportional to the quantity of Tau [pS96] proteins in the brain tissue homogenates. As shown in FIG. 1 administration of LCB 03-0110 at a dosage of 10 mg/kg or less reduces the level of phosphorylated Tau in the APP mice. FIGS. 2A-2B shows that administration of LCB 03-0110 at a dosage of 10 mg/kg or less reduces the ratio of Tau (phosphorylated Tau (pS396)/total Tau)) in the APP mice. However, administration of LCB 03-0110 at a dosage of 10 mg/kg or less does not alter total Tau levels in 12-15 month old APP mice after one week (see FIG. 2C). Surprisingly, lower dosages of LCB 03-0110, i.e., less than 2.5 mg/kg, were more effective in reducing the ratio of Tau in the APP mice than higher dosages of LCB 03-0110, i.e, greater than 5.0 mg/kg. All statistics were performed using ANOVA with Tukey multiple comparison post-test and data were expressed as Mean±SD.

Western Blotting:

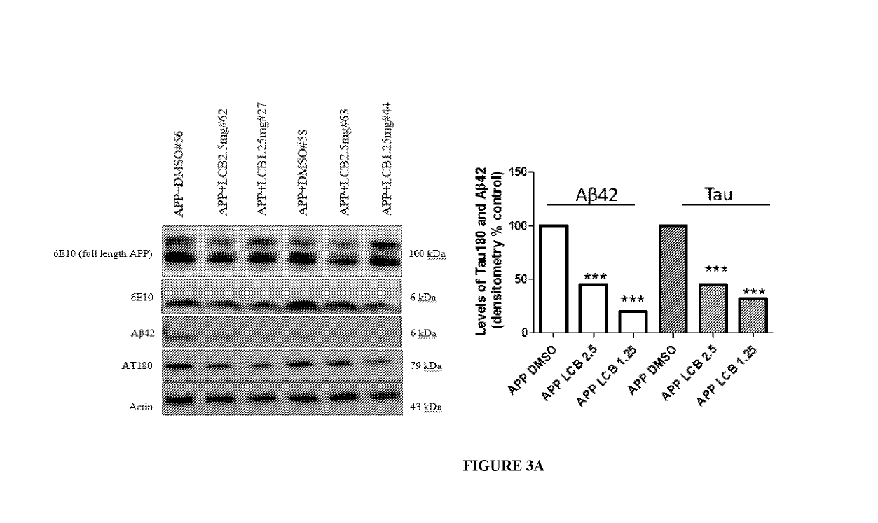

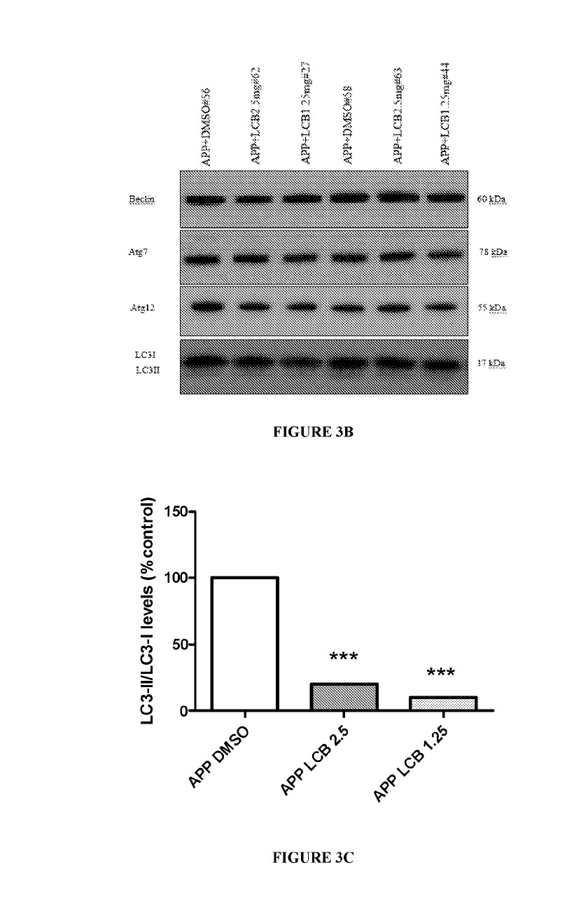

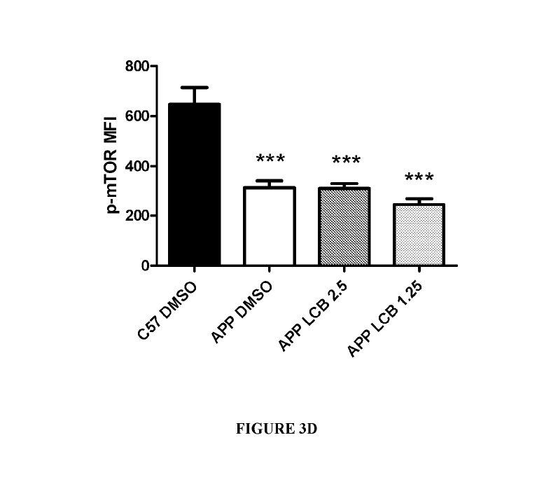

Brain tissues from transgenic APP mice treated with LCB 03-0110 at 10 mg/kg, 5 mg/kg, 2.5 mg/kg, and 1.25 mg/kg were homogenized in 1×STEN buffer, centrifuged at 10,000×g for 20 min at 4° C., and the supernatants containing the soluble fraction were collected. Soluble fractions were probed with mouse monoclonal anti-6E10 (1:1000), rabbit polyclonal anti-Ab42 (1:1000), mouse monoclonal anti-AT180 (1:1000), rabbit polyclonal anti-Beclin-1 (1:1000), rabbit polyclonal anti-Atg7 (1:1000), rabbit polyclonal anti-Atg12 (1:1000), rabbit polyclonal anti-LC3B (1:1000) and rabbit polyclonal anti-actin (1:1000). See FIGS. 3A and 3B. As shown in FIG. 3A, LCB 03-0110 was effective in reducing beta-amyloid (Aβ₄₂) levels (left panel). Densitomety analysis (n=5) shows that LCB-03-110 significantly reduced the levels of amyloid in 3 mutant APP mice that express both Ab42 and hyperphosphorylated tau as early as 4 months of age (right panel). These data indicate that LCB-03-110 can reduce the level of amyloid proteins at concentrations of about 2.5 mg/kg or less.

Administration of LCB-03-0110 (FIG. 3B) stimulates autophagy and clears autophagosomes without altering mTOR (FIG. 3D). Densitometry analysis of LC3-II levels (n=5) showed that LCB-03-110 significantly reduces the level of LCB-II/LCB-I suggesting that it activates autophagic clearance (FIG. 3C). These data indicate that low doses, for example, doses of 2.5 mg/kg or less, of LCB-03-0110 can induce autophagy and clear beta-amyloid and tau, as shown in FIG. 3A.

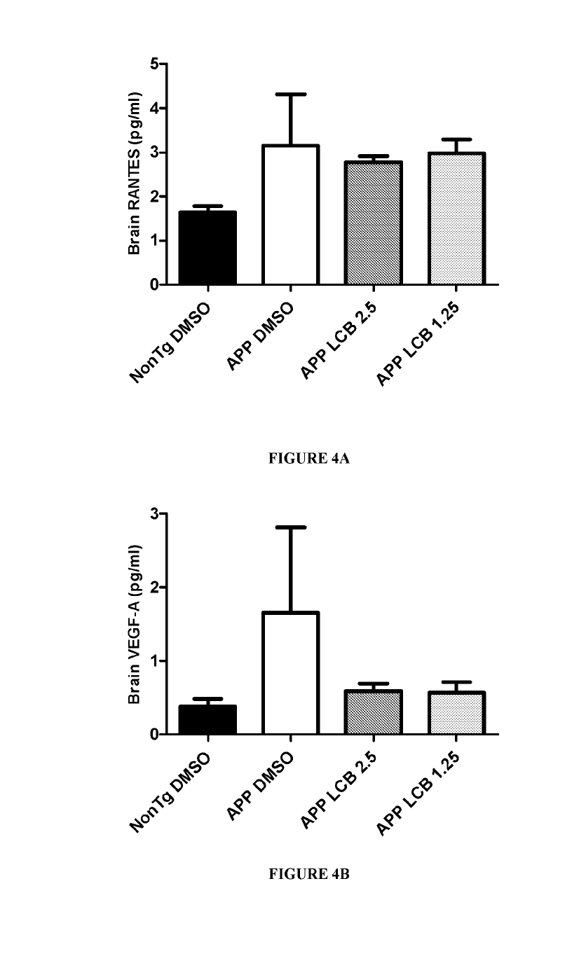

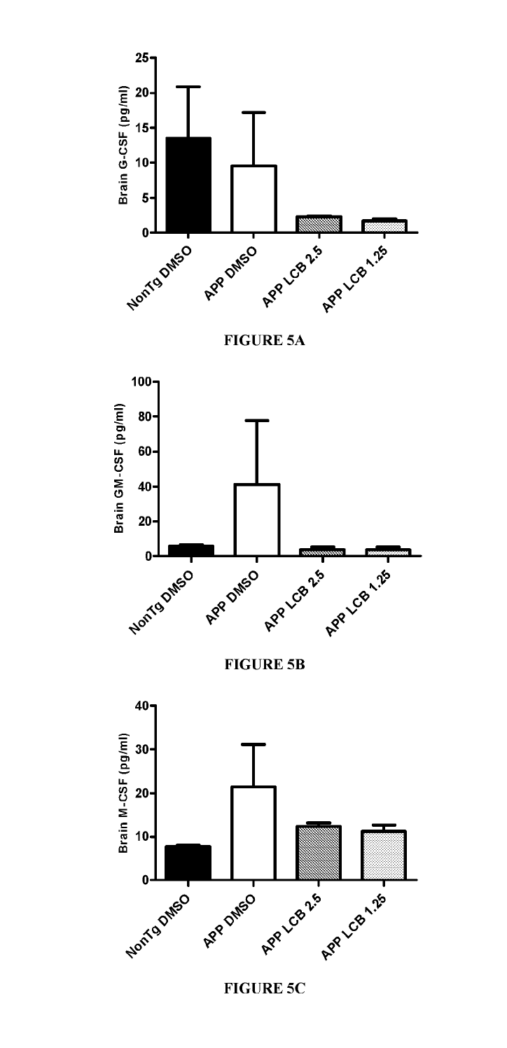

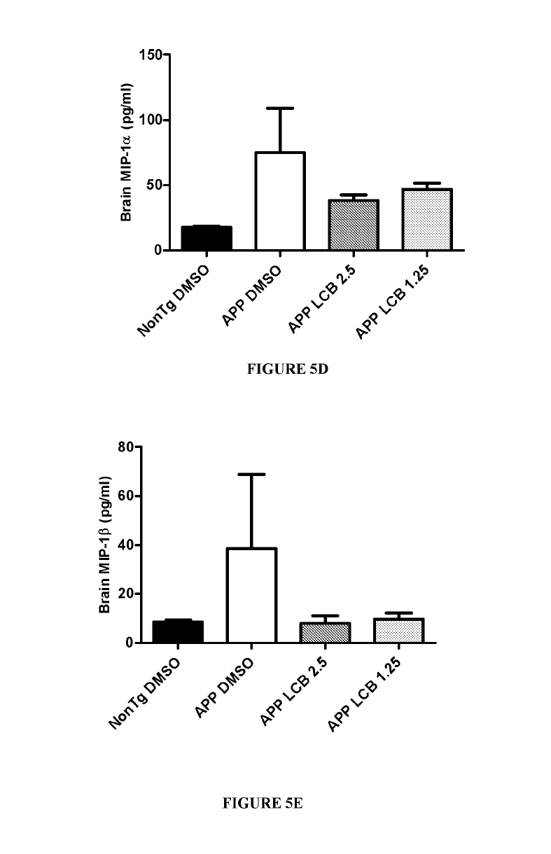

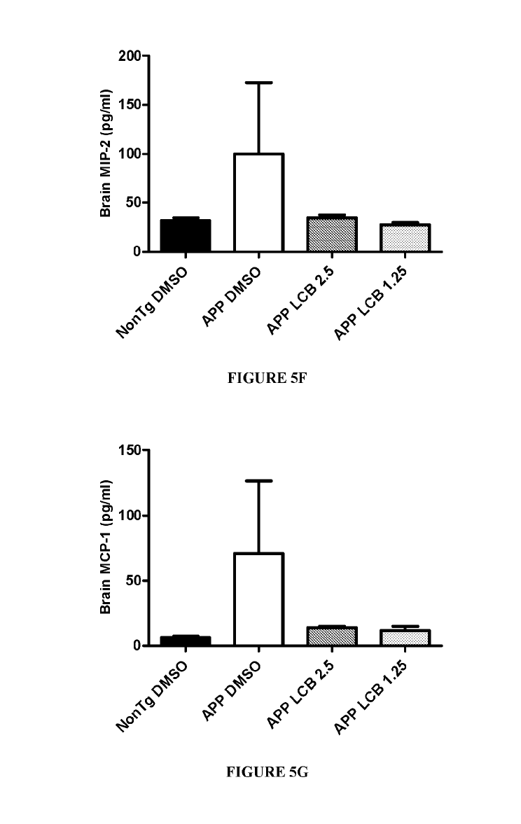

MILLIPLEX®: Cytokines and chemokines were measured in serum and brain with the Mouse Cytokine/Chemokine Magnetic Bead Panel (Cat MCYTOMAG-70K, EMD Millipore). Human amyloid beta and total tau was measured in brain using the Human Amyloid Beta and Tau Panel (Cat HNABTMAG-68K, EMD Millipore). AKT/mTOR signaling cascade phosphoproteins were measured using the Akt/mTOR Phosphoprotein Magnetic Bread 11-Plex Kit (Cat 48-611MAG, EMD Millipore). Assays were performed according to the manufacturer's protocols. As shown in FIGS. 4A and 4B, respectively, administration of 2.5 mg/kg or 1.25 mg/kg of LCB-03-0110 does not alter RANTES levels, but reduces VEGF-A to control levels. Administration of 2.5 mg/kg or 1.25 mg/kg of LCB-03-0110 also reduced brain inflammation, as evidenced by reductions in granulocyte colony-stimulating factor (G-CSF) (FIG. 5A), granulocyte-macrophage colony-stimulating factor (GM-CSF)(FIG. 5B), macrophage colony-stimulating factor (M-CSF) (FIG. 5C), macrophage inflammatory factor 1 alpha (MIP-1α) (FIG. 5D), macrophage inflammatory factor 1 beta (MIP-1β) (FIG. 5E), macrophage inflammatory protein 2 (MIP-2) (FIG. 5F) and macrophage inflammatory protein 1 (MIP-1) (FIG. 5G).

Morris Water Maze:

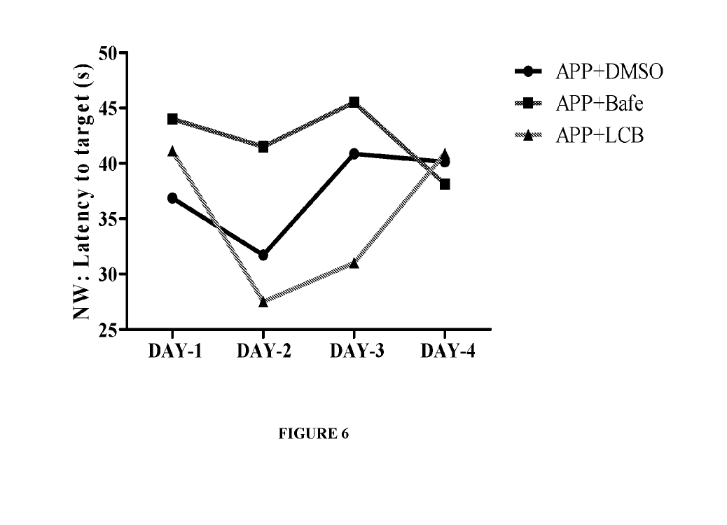

APP mice treated with either DMSO or 2.5 mg/kg LCB-03-0110 for 3 weeks were trained three times a day to find a submerged platform using visuospatial cues inside the apparatus. For each of the three daily training sessions, the mouse was placed in a different quadrant, one of the three which did not contain the platform. The mouse was allowed up to 60 seconds to find the platform, after which it would be removed from the apparatus after 5 seconds on the platform. If the animal was unable to find the platform, it was placed on the platform for 20 seconds. FIG. 6 shows that, by day three, mice treated with 2.5 mg/kg LCB-03110 for three weeks had a 30% reduction in the average time to find a submerged platform. Data are shown as an average of three trials.

Pharmacokinetic Analysis:

C57BL/6 mice received a single IP dose of 1.25, 2.5, 5, or 10 mg/kg LCB, and brain and serum were collected at 2, 4, 6, or 8 hours (n=3 per dose and time point). Animals injected with vehicle (DMSO) were used for background subtraction. Drug levels were determined at the Georgetown University Proteomics and Metabolomics Shared Resources (PMSR). Stock solution for LCB-03110 [(approximately 1 mg/mL each) was prepared in methanol/dichloromethane (50:50). The serial dilutions for each of the standards were produced for the study separately in methanol/HPLC grade water (50:50). Preparation of the calibration curve standards and quality samples (QC) was performed by mixing the stock solutions in blank samples. The final calibration concentration for LCB-03110 ranged from 0.1 ng/mL to 100 ng/mL. The QC concentrations were 30 ng/mL, 3 ng/mL and 0.3 ng/mL, respectively. Serum and brain samples were stored at −80° C. and then thawed to room temperature prior to preparation. The thawed serum samples (20 μL) were transfused to a tube containing 100 μL of water. The 500 μL extraction solvent and acetonitrile/methanol (50:50) was added to the sample. The mixture was vortexed and incubated on ice for 20 minutes to accelerate protein precipitation. After incubation, the samples were vortexed again and centrifuged at 13,000 rpm for 20 minutes at 4° C. The supernatant was then collected and transferred to a new tube, was dried using speed vac, and reconstituted in 200 μL of methanol/water (50:50). The mixture was spun again at 13,000 rpm for 20 minutes at 4° C. The supernatant was then collected into a mass spec sample tube cap and run in the mass spectrometer. For brain, a small section of the thawed brain sample from each animal was transferred to a flat bottom tube. 200 μL of methanol/water (90:10) was added, and the tissue was homogenized. Acetonitrile was then added to the mixture facilitating protein precipitation. The mixture was then incubated on ice for 10 minutes. After incubation, the samples were vortexed and centrifuged at 13,000 rpm for 20 minutes at 4° C. The supernatant was then collected and transferred to a new tube, dried using speed vac, and reconstituted in 200 μL of methanol/water (50:50). The mixture was centrifuged at 13,000 rpm for 20 minutes at 4° C. The supernatant was collected into a mass spec sample tube cap and run in the mass spectrometer. The samples were resolved on an Acquity UPLC BEH C18 1.7 m, 2.1×50 mm column online with a triple quadrupole mass spectrometer (Xevo-TQ-S, Waters Corporation, USA) operating in the multiple reaction monitoring (MRM) mode. The sample cone voltage and collision energies were optimized for both analytes to obtain maximum ion intensity for parent and daughter ions using “Intelli Start” feature of MassLynx software (Waters Corporation, USA). The instrument parameters were optimized to gain maximum specificity and sensitivity of ionization for the parent [m/z=438.25] and daughter ions [m/z=357.33]. Signal intensities from all MRM Q1/Q3 ion pairs for analytes were ranked to ensure selection of the most intense precursor and fragment ion pair for MRM-based quantitation. This approach resulted in selection of cone voltages and collision energies that maximized the generation of each fragment ion species. Analysis was performed with a six to eight-point calibration curve, the sample queue was randomized and solvent blanks were injected to assess sample carryover. MRM data were processed using TargetLynx 4.1. The relative quantification values of analytes were determined by calculating the ratio of peak areas of transitions of samples normalized to the peak area of the internal standard.

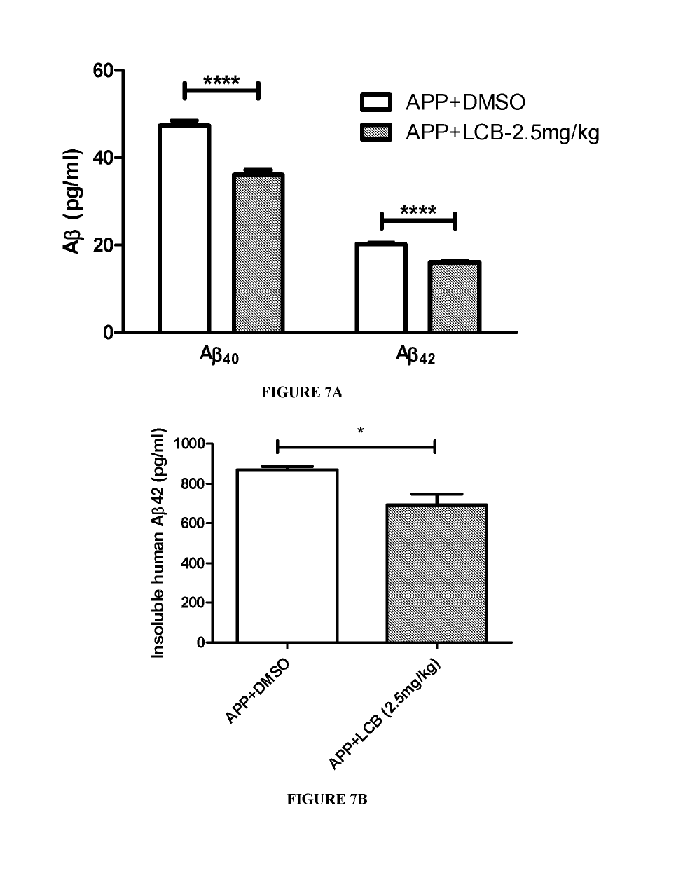

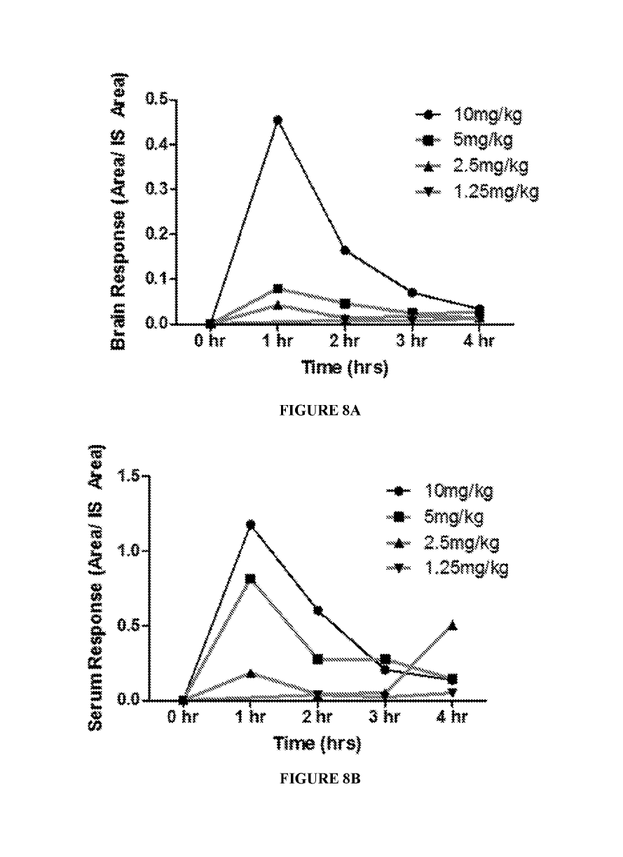

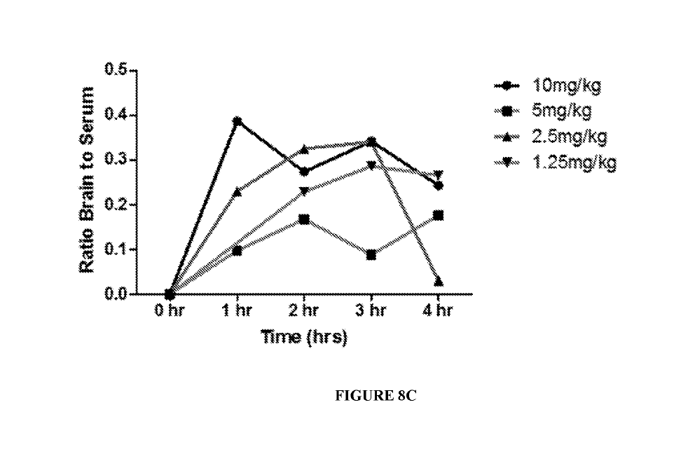

As shown in FIG. 7A and FIG. 7B, respectively, administration of 2.5 mg/kg of LCB-03-0110 significantly reduces levels of soluble and insoluble amyloid beta in the brains of APP mice. As shown in FIG. 8A-8C, LCB-03-0110 penetrates the blood brain barrier. FIG. 8A shows brain response values for LCB-03-0110 in APP mice (Area/IS Area) after administration of 10 mg/kg, 5 mg/kg, 2.5 mg/kg or 1.25 mg/kg LCB-03-0110. FIG. 8B shows serum response values for LCB-03-0110 in APP mice (Area/IS Area) after administration of 10 mg/kg, 5 mg/kg, 2.5 mg/kg or 1.25 mg/kg LCB-03-0110. FIG. 8C shows the ratio of brain to serum response values for LCB-03-0110 in APP mice after administration of 10 mg/kg, 5 mg/kg, 2.5 mg/kg or 1.25 mg/kg LCB-03-0110. Collectively, these data indicate LCB-03-110 peaks in the brain at about one hour and is washed out completely at about four hours (FIG. 8A). In these studies, 10 mg/kg LCB-03-0110 yielded the highest drug concentration in the brain (FIG. 8A), and 2.5 mg/kg LCB-03-110 yielded the highest plasma:brain ratio with a Tmax of about two hours. These data suggest that a dose of 2.5 mg/kg or less can be used to achieve a favorable plasma:brain ratio of LCB-03-0110 for the treatment of neurodegenerative disorders.

What is claimed is:

1. A method of treating or preventing a central nervous system neurodegenerative disease in a subject comprising administering to the subject with the neurodegenerative disease or at risk of developing the neurodegenerative disease an effective amount of a compound having the following formula: 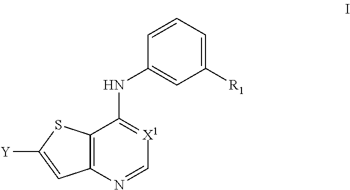
wherein,
X₁ is N or CH;
R₁ is —OH or —OCH₃;
Y is C₆₋₁₀ aryl substituted with R², or C₅₋₁₀ heteroaryl substituted with R² or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, di C₁₋₃ alkylamino, di C₁₋₃ alkylamino, hydroxyl C₁₋₃ alkylamino, carboxy C₁₋₃ alkylamino, C₃₋₆ cycloalkyl C₁₋₃ alkylamino, pyrrolidinyl, hydroxyl pyrrolidinyl, hydroxyl C₁₋₃ alkylpyrolidinyl, carboxypyrrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
or an isomer or pharmaceutically acceptable salt thereof.

2. The method of claim 1, wherein the compound is a compound having the following formula:

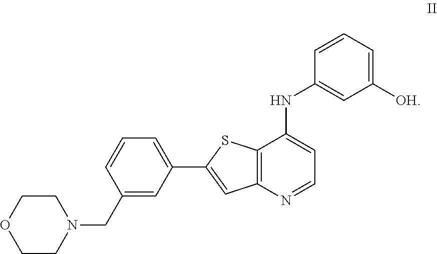 [SMILES: Oc1cccc(Nc2ccnc3cc(-c4cccc(CN5CCOCC5)c4)sc23)c1]

3. The method of claim 1, wherein the compound crosses the blood brain barrier.

4. The method of claim 1, wherein the central nervous system neurodegenerative disease is selected from the group consisting of Amoytrophic Lateral Sclerosis, Alzheimer's Disease, Parkinson's Disease, Huntington's Disease, Mild Cognitive Impairment, an α-Synucleinopathy and a Taupathy.

5. The method of claim 1, wherein the compound is administered systemically.

6. The method of claim 5, wherein the compound is administered orally.

7. The method of claim 1, wherein the dosage is about 10 mg/kg or less.

8. The method of claim 7, wherein the dosage is about 2.5 mg/kg or less.

9. The method of claim 7, wherein the dosage is about 1.25 mg/kg or less.

10. The method of claim 1, wherein the compound is administered daily.

11. The method of claim 1, wherein the compound is in a pharmaceutical composition.

12. The method of claim 1, further comprising administering a second therapeutic agent to the subject.

13. The method of claim 12, wherein the second therapeutic agent is selected from the group consisting of levadopa, a dopamine agonist, an anticholinergic agent, a monoamine oxidase inhibitor, a COMT inhibitor, amantadine, donepezil, memantine, risperidone, rivastigmine, an NMDA antagonist, an acetylcholinesterase inhibitor, a cholinesterase inhibitor, riluzole, an anti-psychotic agent, an antidepressant, and tetrabenazine.

14. A method of inhibiting or preventing toxic protein aggregation in a neuron comprising contacting the neuron with an effective amount of a composition comprising a compound having the following formula: 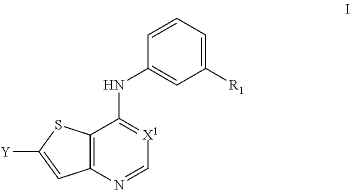
wherein,
X₁ is N or CH;
R₁ is —OH or —OCH₃;
Y is C₆₋₁₀ aryl substituted with R₂, or C₅₋₁₀ heteroaryl substituted with R₂ or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
or an isomer or pharmaceutically acceptable salt thereof.

15. The method of claim 14, wherein the compound is a compound having the following formula:

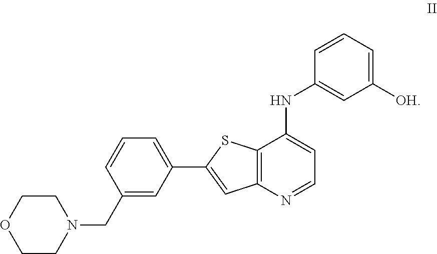 [SMILES: Oc1cccc(Nc2ccnc3cc(-c4cccc(CN5CCOCC5)c4)sc23)c1]

16. The method of claim 14, wherein the protein is selected from the group consisting of an amyloidogenic protein, alpha-synuclein, tau and TDP-43.

17. The method of claim 16, wherein the amyloidogenic protein is β-amyloid.

18. The method of claim 14, wherein the contacting is performed in vivo.

19. The method of claim 14, wherein the contacting is performed in vitro.

20. A method of rescuing a neuron from neurodegeneration comprising contacting the neuron with an effective amount of a composition comprising a compound having the following formula: 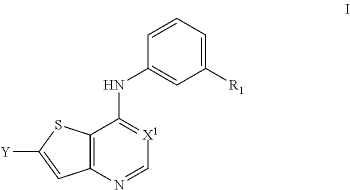
wherein,
X₁ is N or CH;
R₁ is —OH or —OCH₃;
Y is C₆₋₁₀ aryl substituted with R₂, or C₅₋₁₀ heteroaryl substituted with R₂ or N-methylpiperazinyl;
R² is —(CH₂)ₙ—R³, —(CH2)ₙ—C(O)—R³, or —O(CH₂)ₙ—R³;
R³ is —H, —CN, halogen, C₁₋₃ alkyl, C₁₋₃ alkoxy, phenyl, pyridinyl, amino, C₁₋₃ alkyl amino, di C₁₋₃ alkyl amino, hydroxyl C₁₋₃ alkyl amino, carboxy C₁₋₃ alkyl amino, C₃₋₆ cycloalkyl C₁₋₃ alkylamino, pyrrolidinyl, hydroxyl pyrrolidinyl, hydroxyl C₁₋₃ alkylpyrolidinyl, carboxypyrolidinyl, piperidinyl, C₁₋₃ alkylpiperidinyl, di C₁₋₃ alkyl piperidinyl, piperazinyl, C₁₋₃ alkylpiperazinyl, C₁₋₄ alkoxycarbonylpiperazinyl, or morpholinyl; and
n is an integer selected from 0 to 3,
or an isomer or pharmaceutically acceptable salt thereof.

21. The method of claim 20, wherein the compound is a compound having the following formula:

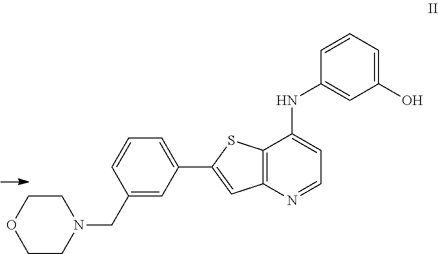 [SMILES: Oc1cccc(Nc2ccnc3cc(-c4cccc(CN5CCOCC5)c4)sc23)c1]

22. The method of claim 20, wherein the contacting is performed in vivo.

23. The method of claim 20, wherein the contacting is performed in vitro.
